(19) United States
(12) Patent Application Publication
(10) Pub. No.: US 2025/0144318 A1
(43) Pub. Date: May 8, 2025

(54) Pen Needle Having a Linear Needle Cover

(71) Applicant: Pentamaster MediQ Sdn Bhd, Simpang Ampat, Pulau Pinang (MY)

(72) Inventors: Seng Chuen CHONG, Penang (MY); Ahmad Fuad Wishnu HUSAIN, Perak (MY)

(73) Assignee: Pentamaster MediQ Sdn Bhd, Simpang Ampat, Pulau Pinang (MY)

(21) Appl. No.: 18/833,408

(22) PCT Filed: Jan. 27, 2023
(86) PCT No.: PCT/MY2023/050005

§ 371 Date: Jul. 26, 2024

(30) Foreign Application Priority Data
Jan. 28, 2022 (MY) PI 2022000539

(51) Int. Cl. A61M 5/32

(52) U.S. Cl. CPC A61M 5/3245; A61M 2005/3254

(57) ABSTRACT

The present invention provides a pen needle (100) having a hub (105) with a first cylindrical compartment and a second cylindrical compartment, wherein the first cylindrical compartment having a smaller circumferential size than the second cylindrical compartment, and a needle (101) held by the hub (105) across the length of the hub (105) defining a center axis, wherein a tip of the needle (101) is exposed within a space of the second cylindrical compartment. The pen needle (100) comprises a spring loaded needle cover (124) installed in the first cylindrical compartment of the hub (105), wherein the spring loaded needle cover (124) comprises a distal end slidable along the center axis to cover the tip of the needle, and two opposingly cantilevered fingers (412, 414), each finger further comprises a protruding peg (422,424) adapted as stopper for restricting movements of the needle cover (124); and a retainer (126) disposed within the second cylindrical compartment and movable linearly in a restricted manner; the retainer (126) comprises a center aperture for receiving the spring loaded needle cover (124).

### FIELD OF THE INVENTION

The present invention relates to pen needle. More specifically, the present invention relates to a single use pen needle with protective feature.

### BACKGROUND

Operating and usage of injection needles or simply needles require great care to protect users and others from needle stick injury. These injuries can occur at any time when people use, disassemble, or dispose of the needles, pre- and post-used of the needles. The greatest concern of such needle stick injuries is the potential for transmitting blood-borne viruses to the others. Therefore, storage and disposal of these needles require special care to reduce the hazards.

For hygiene purposes, needles now are fabricated for single-use only, and the used needles are disposed thereafter. To make it more challenging, needles are no longer only used in a medical care facility, but in a field, and even at home. The needles operators are also not limited to trained medical personnel. Accordingly, it is desired that the needles are design at least to prevent needle stick injuries.

Among other solutions, the most straight forward solution is to cap/cover the needle tips from being reach, especially, post-used.

Pen needle is adapted for attaching to a medication delivery device, such as insulin pens for dispensing medication into human body through the skin. It is desired that the pen needle is adapted to be easily removable and replaceable on the medication delivery device.

For hygiene purposes, any such medical devices should minimize physical contact with the users.

### SUMMARY

In one aspect of the present invention, there is provided a pen needle having a tip of needle held by a hub centrifugally at one end thereof. The pen needle comprises a torsion spring; a pivot rotatably disposed on the hub about the needle, the pivot is engaging with the hub through a torsion spring, wherein the pivot having a ramp, the ramp further include a holding bar projected over the ramp of the pivot; and a slider installed over the hub for covering the tip of the needle, the slider is slidable linearly along a longitudinal of the center axis, with lug defined radially at the proximal end the slider. When the pen needle is assembled, the lug of the slider is parked on the holding bar supporting the slider at a position covering the tip of the needle thereunder; wherein operationally a linear movement of the slider in one linear direction revealed the tip of the needle and the lug urging the ramp to toggle rotation of the pivot to store energy in the torsion spring, and when the stored energy is released, the torsion spring urges the pivot to rotate in reverse to urge the lug lifting the slider back to cover the tip of the needle.

In one embodiment, the pen needle further comprises a channel defined between the ramp and the holding bar, operationally receiving the lug of the slider.

In another embodiment, an end of the channel may comprise a locking platform for holding the lug from moving linearly.

In yet another embodiment, the locking platform is a channel having a perpendicular inclination against center axis.

In a further embodiment, the slider is guided on the hub to move only in a linear movement.

In a further alternative embodiment, the torsion spring, the pivot and the slider are installed centrifugally and externally around the hub.

In another aspect of the present invention, there is provided another pen needle having a bub with a first cylindrical compartment and a second cylindrical compartment, wherein the first cylindrical compartment having a smaller circumferential size than the second cylindrical compartment, and a needle held by the hub across the length of the hub defining a center axis, wherein a tip of the needle is exposed within a space of the second cylindrical compartment. The pen needle comprises a spring loaded needle cover installed in the first cylindrical compartment of the hub, wherein the spring loaded needle cover comprises a distal end slidable along the center axis to cover the tip of the needle, and two opposingly cantilevered fingers, each finger further comprises a protruding peg adapted as stopper for restricting movements of the needle cover; and a retainer disposed within the second cylindrical compartment and movable linearly in a restricted manner; the retainer comprises a center aperture for receiving the spring loaded needle cover. When assembled and unused, the spring loaded needle cover is being restricted by the peg on the finger abutted against the retainer from moving towards the tip of the needle. Operationally, when the retainer is urged by an external force to pushed the needle cover entering the first cylindrical compartment further, the finger is deflected giving clearance to extend through the center aperture of the retainer to cover the tip of the needle when the external forced is removed, while the peg on the finger blocks the needle cover from returning to the first cylindrical compartment to cover the tip permanently.

In one embodiment, the pen needle further comprises a compression spring connecting to the needle cover.

In another embodiment, the pegs are tapered pegs.

In yet another embodiment, the needle cover comprises two sets of opposingly cantilevered fingers cantilevered.

In a further embodiment, wherein the second cylindrical compartment is adapted for receiving a medication delivery device.

In yet a further embodiment, the medication delivery device comprises a spout for coupling with the second cylindrical compartment, wherein when the spout is operationally inserted into and secured with the pen needle, it urges the retainer as the external forces to push the needle cover toward the first cylindrical compartment further.

### BRIEF DESCRIPTION OF THE DRAWINGS

This invention will be described by way of non-limiting embodiments of the present invention, with reference to the accompanying drawings, in which:

FIG. 1 illustrates an explored view of a single-use (or one-time-use) pen needle in accordance with an embodiment of the present invention;

FIG. 2 illustrates a cross sectional view of the pen needle assembled and sealed inside the container in accordance with an embodiment of the present invention;

FIG. 3 illustrates a perspective view of the hub of FIG. 1 of one embodiment of the present invention;

FIG. 4A illustrates a front perspective view of the needle cover of FIG. 1 in accordance with an embodiment of the present invention;

FIG. 4B illustrates a front perspective view of the retainer of FIG. 1 in accordance with an embodiment of the present invention;

FIG. 5 illustrates an operating state of the rear end of the pen needle of FIG. 1 in accordance with an embodiment of the present invention;

FIG. 6A illustrates a closed-up view of the slider of FIG. 1 of one embodiment of the present invention;

FIG. 6B illustrates a perspective view of the pivot of FIG. 1 of one embodiment of the present invention;

FIG. 6C illustrates another perspective views of the pivot of FIG. 6B; and

FIGS. 7A-7C illustrates operating states of the front end of the pen needle of FIG. 1 in accordance with an embodiment of the present invention.

### DETAILED DESCRIPTION

In line with the above summary, the following description of a number of specific and alternative embodiments are provided to understand the inventive features of the present invention. It shall be apparent to one skilled in the art, however that this invention may be practiced without such specific details. Some of the details may not be described at length so as not to obscure the invention. For ease of reference, common reference numerals will be used throughout the figures when referring to the same or similar features common to the figures.

As used in the description of the embodiments and the appended claims, the singular forms “a”, “an” and “the” are intended to include the plural forms as well, unless the context clearly indicates otherwise. The words “and/or” as used herein refer to and encompass any and all possible combinations of one or more of the associated listed items. The words “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The words “right.” “left,” “lower” and “upper” designate directions in the drawings to which reference is made. The words “inwardly” and “outwardly” refer to directions toward and away from, respectively, the geometric center (or axis) of a needle, and designated parts thereof. Whenever the words “front” and “back/rear” are used, unless otherwise stated, the end with the needle for penetrating into the skin shall be taken as front end and the opposing end shall be taken as back (end) or rear end. In any event, these terms are used for case of illustrations only, not a limitations.

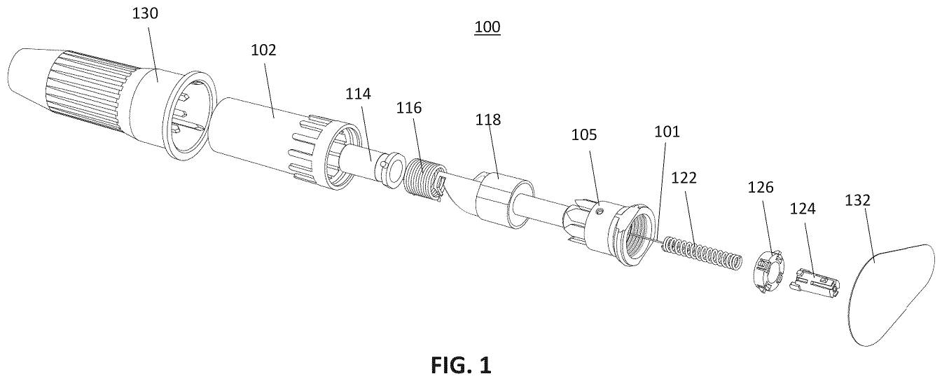

FIG. 1 illustrates an explored view of a single-use (or one-time-use) pen needle 100 in accordance with an embodiment of the present invention. The pen needle 100 is adapted to be disposable after single-use, and needle tips of the pen needle 100 will be covered for disposal. The pen needle 100 comprises a hub 105, a needle 101, a shield 102, a slider 114, a torsion spring 116, a pivot 118, a safety spring 122, a needle cover 124 and a retainer 126, adapted to assemble along a longitudinal axis defined by the length of the needle 101. The pen needle 100, when newly assembled and unused, is sealed within a container 130 with a paper seal 132. User is required to peel off the paper seal 132 to reveal the pen needle 100. Then, the medication delivery device can be inserted to the container 130 and to attach the pen needle 100 thereon while it remains in the container 130.

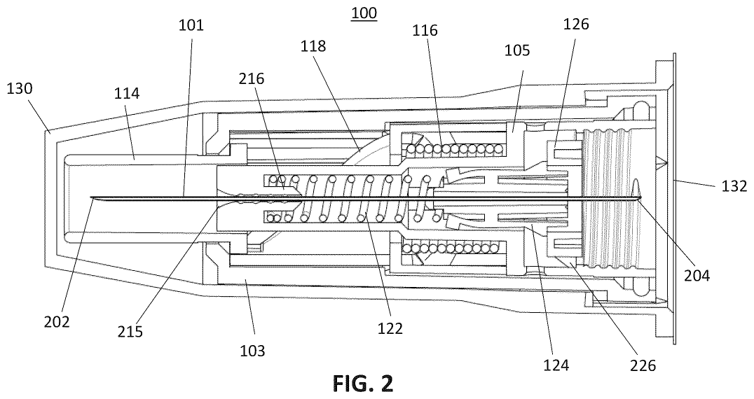

FIG. 2 illustrates a cross sectional view of the pen needle 100 assembled and sealed inside the container 130 in accordance with an embodiment of the present invention. For ease of illustrations, the assembled pen needle 100 shall comprise two distal ends, which are herein referred to as a device end (or rear end) and a skin end (or front end) respectively. In general, the slider 114, the torsion spring 116 and the pivot 118 collectively form the front end (or skin end); the safety spring 122, needle cover 124 and the retainer 126 collectively form the rear end (or device end); and the shield 102, the hub 105 and the needle 101 are disposed as a center hub holding the components of the two distal ends.

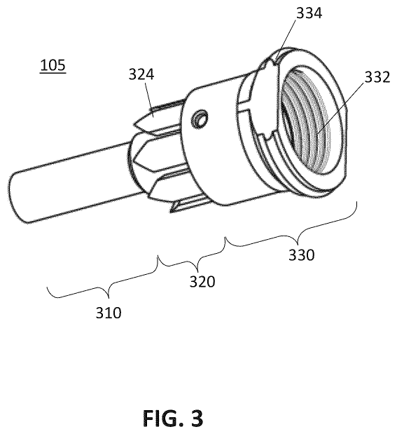

Still referring to FIG. 2, the hub 105 is adapted with profiles and configurations to hold the components forming the pen needle 100. A perspective view of the hub 105 of one embodiment of the present invention is illustrated in FIG. 3. The hub 105 is generally formed as an extended telescopic cylinder with three main sections: a first section 310 disposed at the front end having a smallest diameter; a second section 320 defining the middle section of the hub 105 and a third section 330 forming the rear end with the largest diameter of the extended telescopic cylinder.

From the left side of the figure, the first section 310 of the hub 105 defines a front end of the hub 105 with a smallest circumference. In general, the first section 310 forms a cylinder tube having a front aperture 215 at its front distal end and axially extending in the tube at a small length towards the rear end for receiving and supporting the needle 101. In one embodiment, the front aperture 215 axially extending in the inner side of the first section 310 defining an inner collar 216 which provide a better support to the needle 101.

The second section 320 defines the mid portion of the hub 105 has a relatively larger circumference tube bridging the first section 310 and the third section 330. The second section 320 comprises guiding channels 324 formed and arranged around circular exterior thereon, and a smooth interior surface thereunder. It is understood to a skilled person that these guiding means are facilitated to assist installation and assembly to put the components in a desired orientation and operations.

The third section 330 defines the rear end of the hub 105. In the inner surface towards a proximal rear end of third section 330, it further defines internal threads 332 for attaching to the medication delivery devices. In one embodiment, the internal threads shall be made to comply with at least ISO 11608-2:2012. The terminal rear end of the hub 105 further defines a protruding rim 334.

Referring back to the needle 101, in one embodiment, the aperture 215 is adapted to hold the needle 101 along the center axis of the pen needle 100. Preferably, the aperture is sized to snugly fits and holds the needle 101 in place. In one embodiment, it is desired that the needle is assembled to have a fixed position and orientation relative to the hub 105. The front tip 202 of the needle 101 extends beyond the first section 310 of the hub 105, with the needle body runs along the center axis towards the rear end thereof and the rear tip 204 terminates at a short distance before the rear end rim of the hub 105. In yet another embodiment, the needle 101 is defined under ISO 9626:2016.

Referring now to the rear end components, i.e. the spring 122, the needle cover 124 and the retainer 126. The spring 122 is inserted from the rear end of the hub 105 all the way into the inner space of first section 310 around the needle 101 in the hub 105. The front end of the spring 122 is fitted into a ring channel between the inner collar 216 and an outer wall of the first section 310. The length of the spring 122 is extended into the second section 320 of the hub 105.

Following the spring 122, the needle cover 124 is inserted into the hub 105 followed by the retainer 126. The needle cover 124 is adapted to fit into the second section 320 of the hub 105 with its rear end disposed in the third section 330 of the hub 105. When the needle cover 124 is inserted into the second section 320, it abuts and then compresses the spring 122. The retainer 126 is further inserted into the hub 105 through the third section 330 for holding the needle cover 124 in place such that the spring 122 remains compressed. The retainer 126 adapted with tapered pegs 226 for engaging with interior profiles in the third section 330 in a manner that the interior profiles limit the retainer 126 to back up exiting the hub 105. When the retainer 126 is in place in the hub 105, the retainer 126 urges the needle cover 124 to remain compressing the spring 122. Within the third section 330, the retainer 126 is still movable in a reciprocal manner within the third section 330. And further when the retainer 126 is in place, the rear end of the needle tip 204 is exposed in the third section 330.

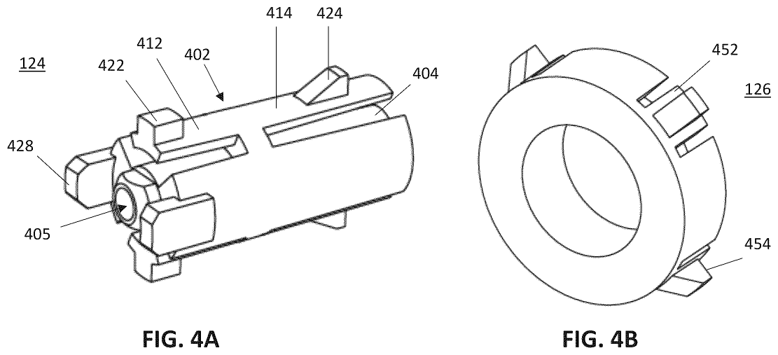

FIG. 4A illustrates a front perspective view of the needle cover 124 in accordance with an embodiment of the present invention. The needle cover 124 is generally a cylindrical tube 402 having a concentric inner tube 404. The concentric inner tube 404 provides a cylindrical path 405 for receiving the needle 101 when inserted into the hub 105. The inner diameter of the concentric inner tube 404 is larger than that of the needle 101 such that the needle cover 124 has free movements against the needle 101.

A plurality of fingers 412, 414 are defined and arranged radially on the cylindrical tube 405 with their distal end suspend above the concentric inner tube 404. In such manner, the distal ends of the fingers are bendable towards the inner tube 404 when receiving external forces thereon and bounce back when the exerting force is removed. At or near the distal end of each finger 412, 414 is a free end and it defines outwardly projected pegs 422, 424. The free ends of the fingers 412 are pointing towards the front end while the fingers 414 are pointing toward the rear ends. The pegs 422, 424 is provided as snap-on mechanism for engaging and disengaging of the components.

In one specific embodiment, the pegs on the fingers 414 is tapered toward the front end such that when the needle cover 124 is forced toward the second section 320 of the hub 105 further, it depresses and bends the fingers 414 allowing the needle cover 124 to be fully inserted into the second section 320.

The guides 428 are provided for ensuring the position and orientation during assembly and operations. guides 428 are designed specific to fit the desired functionalities of the pen needle 100. Shapes and configurations can be in variant according to the actual designs.

FIG. 4B illustrates a front perspective view of the retainer 126 in accordance with an embodiment of the present invention. The retainer 126 is formed generally as a ring with fingers 452 radially arranged around the circumferential side of the retainer 126. Similarly, the end of the fingers 452 too defines tapered pegs 454.

The general outside diameter of the retainer 126 is adapted to be able to be inserted into the third section 330 of the hub 105. The inner diameter of the retainer 126 is adapted to accommodate the diameter of the outer diameter of the needle cover 124, without the pegs 422, 424. In one embodiment, the inner diameter of the retainer 126 is adapted to about the same or substantially the same as the inner diameter of the second section 320 of the hub 105.

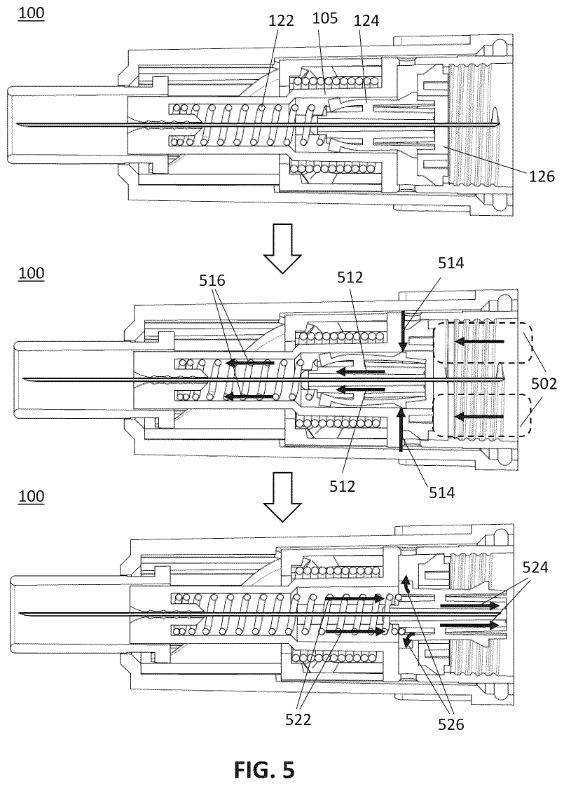

FIG. 5 illustrates an operating state of the rear end of the pen needle in accordance with an embodiment of the present invention. The figure at the top of FIG. 5 shows the pen needle 100 at its assembled, but unused state. To clarify, the rear end of is at an unused state when it has not been connected to any medication delivery device. At this stage, from front to rear end, the spring 122, the needle cover 124 and the retainer 126 are arranged in that sequence within the hub 105. The spring 122 is installed at the front end in the hub 105, compressed by front end of the needle cover 124 as the front end is disposed within the second section of the hub 105. With the front end of the needle cover 124 inserted into the second section of the hub 105, the protruding pegs 422 on the fingers 412 at the front end the needle cover 124 caused the fingers to deflect inwardly towards the center axis so that it can be inserted into the second section of the hub 105. The rear end of the needle cover 124 is exposed within the third section of the hub 105. The needle cover 124 is further held in place by the pegs 454 of the retainer 126 in the hub 105. At this stage, the spring 122 urges the needle cover 124 against the retainer 126 towards the rear end, whereby the tapered pegs 424 served as a stopper to prevent the needle cover 124 to pass through the retainer 126, hence the tip of the needle 101 remains exposed.

When the pen needle 100 is in used, it is attached to a medication delivery device 502 as shown in the second figure of FIG. 5. As the spout of the medication delivery device 502 advances into the hub 105, it urges the retainer 126 which in turn pushes the needle cover 124 further into the second section of the hub 105. As the needle cover 124 advances into the hub 105 as arrows 512, the tapered pegs 424 on the needle cover 124 causes the fingers 414 to deflect inwardly toward the center axis as shown by arrows 514. At this stage, the fingers 412, 414 are all deflected with the pegs 422, 424 cleared from obstructing the needle cover 124 from entering the second section 320 of the hub 105. That in turn compresses the spring 122 further as shown by the arrows 516. When the fingers 414 with tapered pegs 424 are deflected to allow the needle cover 124 to enter the second section 320 of the hub 105, the spring 122 pushes the needle cover 124 in reverse direction to slip through the center hole of the retainer 126. At this stage, the exposed tip of the needle 101 penetrates into the medication delivery device 502 for receiving the medication.

When the medication delivery device 502 is detached from the pen needle 100, without the spout of the medication delivery device 502 holding the parts, the needle cover 124 is freed and the spring 122 pushes the rear end of the needle cover 124 through the retainer 126. As the spring 122 pushes the needle cover 124 out from the second section 320 of the hub 105 (as shown by the arrows 524), the fingers 412 bounces back 105 as shown by the arrows 526 with the pegs 422 permanently blocking the needle cover 124 from returning into the second section 320 of the hub 105. The length if the needle cover 124 is adapted to be long enough to hide the tip of the needle 101 within the inner cylinder 404 within the path 405, hence the rear end tip of the needle 101 is no longer exposed and accessible to user after used.

The rear end of the pen needle 100 provide a configuration and mechanism that covers the tip of the needle 101 automatically after single used. As shown above, user needs to only peel off the top cover of the container then to attach the medication delivery device to the pen needle 100 without physically touching the pen needle 100. When the pen needle 100 is detached from the medication delivery device and the top at the rear end of the needle 101 is covered seamlessly without any extra effort needed from user. In this embodiment, the needle cover 124 provides a linear cover for covering the tip of the needle 101 after single use.

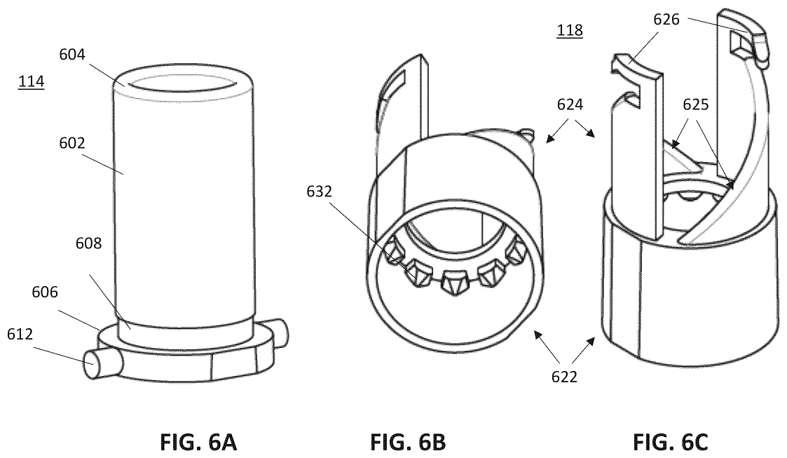

Now referring to the front end of the pen needle 100, which comprises the slider 114, the torsion spring 116 and the pivot 118. The components of the front end of the pen needle 100 are installed over the exterior of the hub 105 and ultimately secured by the shield 102. FIG. 6A illustrates a closed-up view of the slider 114 of one embodiment of the present invention. FIG. 6B and FIG. 6C illustrate two-perspective views of the pivot 118 of one embodiment of the present invention. The front end of the pen needle 100 uses the slider 114 to generally cover the front tip of the needle 101, and only exposed it when it is in used, i.e. penetrating through the skin. The pivot 118 is adapted to support actions of the slider 114 and locks it in place after a single use.

Referring now to FIG. 6A, the slider 114 comprises a cylindrical tube body 602 with one opened end. The opened end defines a rounded lip which is gentle to skin contact. The opposing end of the cylindrical tube body 602 defines a flange 606, which adapted as a stopper for holding the slider in place. Over the circumferential surface of the flange 606, there are lugs 612 extending away from the center axis. The lugs 612 is adapted to engage and trigger a movement of the pivot 118. Further, the cylindrical tube 602 defines a ring channel 608 next to the flange 606, adapted as an after-use indicator. In one embodiment, it can be color coded.

Referring now to FIG. 6B and FIG. 6C, the pivot 118 comprises a cylindrical tube 622 and a guided lock 624 radially formed along the top rim of the cylindrical tube 622 about the center axis. More specifically, the cylindrical tube 622 is sized to cover around the second section 320 of the hub 105 externally. The inner circular surface of the pivot 118 defines guiding channels 632 for engagement with the torsion spring 116. The guided lock 624 comprises a pair of ramps 625, whereby when an external force is acted against the ramps 625, the pivot 118 rotates. The guide lock 624 further comprises a holding bar 626 projected over a higher side of each ram 625. The holding bars 626 are provided for holding the slider 114 thereon. The gap between each the holding bar 626 and the ramp 625 forms a retaining channel for receiving the lug 612 of the slider 114. When the lugs 612 enters the retaining channels of the guided lock 624, the lugs 612 are retained in place causing the slider 114 locks in place permanently, hence single-use.

Referring back to FIG. 2, when the slider 114 and the guided pivot 118 are installed over the hub 105, the cylindrical tube 622 is covering the hub 105 with a space between for accommodating the torsion spring 116. The torsion spring 116, when installed, is engaged with the cylindrical tube 622 at one end and the other end engages with the hub 105 body through the guiding channels 632 and 324. The guided lock 624 formed on the cylindrical tube 622 is provided as a trigger for the radial movements of the pivot 118 relative to the hub 105 in one radial direction. The torsion spring 116 is adapted to engaged with the hub 105 and the pivot 118 to bias in the other radial direction under stress.

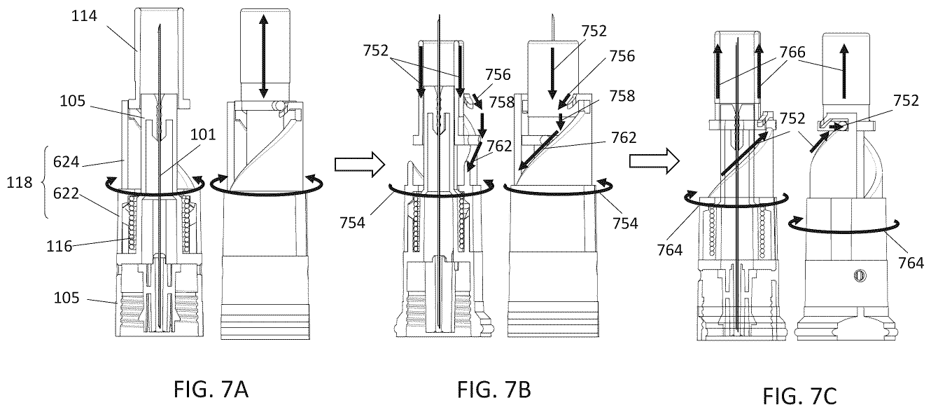

FIG. 7A, FIG. 7B and FIG. 7C shows the operating states the operating states In these figures, the shield of the pen needle 100 is being omitted to simply the illustrations. When assembled, the shield 102 of the pen needle 100 remains as part of the pen needle 100 to hold the parts in place. According to one embodiment of the present invention, once the pen needle 100 is assembled as one piece, no components will be separated from the main body. In each figure, it provides a crossed sectional view and a side exterior view of the pen needle 100.

Referring now to FIG. 7A, the slider 114 is adapted to be moveable linearly along the center axis and the pivot 118 is adapted to be rotatable about the center axis relative to the hub 105. As shown, the torsion spring 116 is further connecting between the pivot 118 and the hub 105, whereby the radial movement of the pivot 118 (relative to the hub 105) in one direction stores energy in the torsion spring 116 and when the torsion energy is released, the energy actuates the pivot 118 in an opposing direction.

In the current embodiment, the slider 114 is restricted from any radial movements operationally and similarly the pivot 118 is restricted from any linear movement on the hub 105.

In FIG. 7A, the slider 114 is held at its highest position by the holding bar 626 and when the slider 114 is at its highest position, the tip at the front end of the needle 101 is being covered under the slider 114. As further illustrated, the holding bar 626 has a tapered end inclined towards the ramp at a short distance. The lugs of the slider 114 are parked on the tapered end to maintain in that highest position. This is the state when the pen needle is not yet been used.

In FIGS. 7A, 7B and 7C, the pen needles 100 are oriented in an upright manner having it front end on top and the rear end at the bottom. Accordingly, the term “top”, “bottom”, “up”, “upward”, “down” and “downward” shall designate directions and position as illustrated in the figures.

Referring now to the FIG. 7B that show that the slider 114 is being push down to expose the tip of the needle 101. When an external force is applied onto the slider 114 (arrows 752), the lugs parked on the tapered ends of the holding bar 626 act a downward force to rotate (arrow 754) the pivot 118. As the pivot 118 rotates (arrow 754), the lugs slide down (arrow 756) and then drop off (arrow 758) from the tapered ends onto the ramp. As the slider 114 is acting downward further, the pivot 118 rotates further allowing the lugs to slide down further on the ramp (arrow 762). The radial movement of the pivot 118 is toggled by the downward moving slider 114 against the ramp. In this embodiment, the direction of the ramp is tapered causes the pivot 118 to rotate in a counterclockwise direction when the slider is moving downward to reveal the needle tip. As the pivot 118 is turning in counterclockwise direction, the torsion spring 116 accumulates more energy therein.

The FIG. 7C illustrates the pen needle 100 when the slider 114 is released to cover the tip of the needle 101. When the slider 114 is freed from any external forces, the torsion spring 116 releases energy stored thereon to toggle the pivot 118 to rotate (arrow 764) in a clockwise direction. That in turn trigger the lugs of the slider 114 along the ramp to lift (arrow 766) the slider 114 up to cover the tip of the needle 101. As the pivot 118 turns, the lugs advance into the channels between the holding bars and the ramps respectively. To further secure the lugs, the end of the channels defined a locking platform, which is simply a relatively flatter channel then the ramp inclination. Preferably, the channel is perpendicular to the center axis, which is also the direction of the movements of the slider 118.

Further in accordance with the embodiments of the present invention, as the front end (except the body of the slider 114) of the pen needle 101 is mostly covered under the shield 103, one can't reverse the action.

In the present invention, the spring 122 may include a compression spring, or any flexible or resilient members adapted to engage and to force the needle cover in a lateral direction. The spring can be fabricated with any suitable material, which include plastic material. Similarly, the torsion spring 116 can also be made by any suitable resilient materials, which include plastic materials.

Further in the present invention, the pegs, lugs are referring to protrusion from a surface, usually outwardly protruded, which can be in any suitable shapes and configurations depending on its application. It a squarish block, or block with tapered surface or rounded surfaces, when applicable. A person skilled in art would appreciate the use of these pegs, lugs in conjunction with flexible fingers to achieve the objectives of the present invention. In the present invention, the inventors make use of internal and external profiles of the parts to work in correspond with another to achieve the objectives. Through the configurations, the pen needle 100 can be attached to a medication delivery device when it is still within the container 130; remove the pen needle 100 from the container 130 through the medication delivery device; penetrate the skin with the needle 101 of the pen needle 100 by pressing the slider 114 against the skin; remove the needle 101 from skin whereby the front tip of the needle is automatically covered under the slider 114; then remove the pen needle 100 from the medication delivery device whereby the rear tip of the needle is automatically covered upon removal; and disposed the pen needle 100 with both end tips of the pen needle 100 covered. All are done with a seamless flow with minimal contact with the pen needle 100 up under it is removed from the medication delivery device. Further, the present invention depends on mechanical means for automatically covering the tips of the needle 101, and there is no electricity require to operate the pen needle 100.

While specific embodiments have been described and illustrated, it is understood that many changes, modifications, variations and combinations thereof could be made to the present invention without departing from the scope of the invention.

1. A pen needle (100) having a hub (105) with a first cylindrical compartment and a second cylindrical compartment, wherein the first cylindrical compartment having a smaller circumferential size than the second cylindrical compartment, and a needle (101) held by the hub (105) across the length of the hub (105) defining a center axis, wherein a tip of the needle (101) is exposed within a space of the second cylindrical compartment, the pen needle (100) comprising:
a spring loaded needle cover (124) installed in the first cylindrical compartment of the hub (105), wherein the spring loaded needle cover (124) comprises a distal end slidable along the center axis to cover the tip of the needle, and two opposingly cantilevered fingers (412, 414), each finger further comprises a protruding peg (422,424) adapted as stopper for restricting movements of the needle cover (124); and
a retainer (126) disposed within the second cylindrical compartment and movable linearly in a restricted manner; the retainer (126) comprises a center aperture for receiving the spring loaded needle cover (124), wherein when assembled and unused, the spring loaded needle cover (124) is being restricted by the peg (424) on the finger (414) abutted against the retainer (126) a the from moving towards the tip of the needle (101); and
operationally, when the retainer (126) is urges by an external force to pushed the needle cover (124) entering the first cylindrical compartment further, the finger (414) is deflected giving clearance to extend through the center aperture of the retainer (126) to cover the tip of the needle (101) when the external forced is removed, while the peg 422) on the finger (412) blocks the needle cover (124) from returning to the first cylindrical compartment to cover the tip permanently.

2. The pen needle (100) according to claim 1, further comprises a compression spring (122) connecting to the needle cover (124).

3. The pen needle (100) according to claim 1, wherein the peg (424) are tapered pegs (424).

4. The pen needle (100) according to claim 1, the needle cover (124) comprises two sets of opposingly cantilevered fingers (412, 414) cantilevered.

5. The pen needle (100) according to claim 1, wherein the second cylindrical compartment is adapted for receiving a medication delivery device.

6. The pen needle (100) according to claim 5, wherein the medication delivery device comprises a spout for coupling with the second cylindrical compartment, wherein when the spout is operationally inserted into and secured with the pen needle (100), it urges the retainer (126) as the external forces to push the needle cover (124) toward the first cylindrical compartment further.
